(19) United States
(12) Patent Application Publication
(10) Pub. No.: US 2017/0252248 A1
(43) Pub. Date: Sep. 7, 2017

(54) TOOL MANIPULATOR AND SYSTEM FOR POSITIONING A TOOL FOR SURGICAL AND LIKE USES

(71) Applicant: UMANO MEDICAL INC., L'lslet (CA)

(72) Inventors: Jean-Sebastien PLANTE, Sherbrooke (CA); Genevieve MIRON, Sherbrooke (CA); David BOUCHARD, Sherbrooke (CA); Jerome MARCOTTE, St-Jean-Chrysostome (CA); Richard LABBE, Sainte-Marie-de-Beauce (CA); Sylvain LACASSE, St-Nicolas (CA); Claudia CHEVREFILS, Montreal (CA); Louis-philippe DION, Quebec (CA); David Mayen MORENO, Quebec (CA); Mathieu MERCIER, Levis (CA)

(21) Appl. No.: 15/514,031

(22) PCT Filed: Sep. 24, 2015
(86) PCT No.: PCT/CA2015/050946

§ 371 Date: Mar. 24, 2017

Related U.S. Application Data
(60) Provisional application No. 62/054,533, filed on Sep. 24, 2014.

(51) Int. Cl. A61G 13/12; A61B 90/11; A61G 13/10; A61B 34/00

(52) U.S. Cl. CPC A61G 13/12; A61B 34/70; A61B 90/11; A61G 13/125; A61G 13/101; A61B 2017/00274

(57) ABSTRACT

The present disclosure relates to a tool manipulator, comprising a base, mountable on an operation table. A caliper is supported by the base and a tool holder is mounted on the caliper. An actuator positionable below a patient supporting surface of the operation table receives positioning commands for moving a tool in at least three degrees of freedom. The tool manipulator can be made part of a system for positioning a needle for diagnosis or treatment of the prostate of a patient. The system also comprises a power source connected to the actuator and a controller controlling the provision of the positioning commands to the actuator.

### TECHNICAL FIELD

The present disclosure relates to the field of precision devices and systems. More specifically, the present disclosure relates to a tool manipulator and to a system for positioning a tool for surgical and like uses.

### BACKGROUND

Prostate cancer affects one out of every eight (8) male adults in North America and is a significant cause of death for elderly men. Besides cancer, other health problems related to the prostate are common and include for example benign prostatic hyperplasia.

Diagnosis of prostate ailments as well as treatment of the prostate are conventional medical procedures. It is common to use medical imaging techniques to guide a clinician in inserting needles within the prostate of a patient under local or general anesthesia, usually through the perineum, to obtain a biopsy of the prostate, to deliver a low-dose or high-dose radiation brachytherapy treatment, and the like.

Conventional systems, such as those using a brachytherapy template to guide transperineal needle insertion in the prostate, are unstable, bulky, and imprecise. They are difficult to register to medical imaging systems and not appropriately designed for multi-trajectory needle insertion. These drawbacks cause significant inconvenience to clinicians, increasing the time required to set up the patient and to perform such medical procedures. These drawbacks may also impair safe and effective procedures in challenging cases.

Recent robotic manipulators have been proposed to circumvent these limitations. However these systems are still excessively bulky, require significant setup time, and in many cases fail to provide full multi-trajectory needle insertion capability. Moreover, these systems preclude the use of an endorectal antenna or coil required for high-resolution magnetic resonance imaging acquisition. As a result, these medical interventions—which will become increasingly common given the aging of the population in developed countries—will continue to suffer from deficiencies in terms of operational effectiveness.

Therefore, there is a need for devices and systems helping in the manipulation of needles for diagnosis and treatment of the prostate of a patient with limited bulk and inconvenience to clinicians. Such devices and systems should also be adaptable for other uses that require fine positioning of tools, for example elongated tools.

### SUMMARY

The present disclosure provides a tool manipulator, comprising a base, a caliper, a tool holder and an actuator. The base is configured for mounting on an operation table. The caliper is supported by the base and the tool holder is mounted on the caliper. The actuator is positionable below a patient supporting surface of the operation table. The actuator is configured to receive positioning commands for moving a tool in at least three degrees of freedom.

According to the present disclosure, there is also provided a system for positioning a needle for diagnosis or treatment of the prostate of a patient. The system comprises an operation table and a tool manipulator having a base, a caliper supported by the base, a tool holder mounted on the caliper, and an actuator positionable below a patient supporting surface of the operation table, the actuator being configured to receive positioning commands for moving a tool in at least three degrees of freedom. The tool manipulator is adapted to support a needle and is integrated in the operation table. The system also comprises a power source operably connected to the actuator, and a controller operably connected to the power source and controlling the provision of the positioning commands to the actuator.

The foregoing and other features will become more apparent upon reading of the following non-restrictive description of illustrative embodiments thereof, given by way of example only with reference to the accompanying drawings.

### BRIEF DESCRIPTION OF THE DRAWINGS

Embodiments of the disclosure will be described by way of example only with reference to the accompanying drawings, in which:

FIG. 1 is a top perspective view of a needle manipulator according to a first embodiment;

FIG. 2 is a bottom perspective view of the needle manipulator of FIG. 1;

FIG. 3 is a perspective view of the needle manipulator of FIG. 1 showing a detail of a pneumatic brake;

FIG. 4 is a top perspective view of a needle manipulator according to a second embodiment;

FIG. 5 is a bottom perspective view of the needle manipulator of FIG. 4;

FIG. 6 is a rear elevation view of the needle manipulator of FIG. 4;

FIG. 7 is a perspective view of a system for positioning a needle for treatment of the prostate of a patient according to a first embodiment;

FIG. 8 is an exploded view of the system of FIG. 7;

FIGS. 9a-9d are detailed views of footrests of the system of FIG. 7, showing their adjustability over four (4) degrees of freedom;

FIG. 10 is a rear perspective view of a needle manipulator, shown without a cover, according to a third embodiment;

FIG. 11 is a front perspective view of the needle manipulator of FIG. 10, shown with a cover;

FIG. 12 is a bottom perspective view a upper movable base and of a needle support of the needle manipulator of FIG. 10;

FIG. 13 is a top perspective view of the upper movable base and of the needle support of FIG. 12;

FIG. 14 is a bottom plan view of the needle manipulator of FIG. 10, shown without a cover;

FIG. 15 is a rear perspective view of a upper movable base and of a needle support of a fourth embodiment of a needle manipulator;

FIG. 16 is a top view of the upper movable base of FIG. 15;

FIG. 17 is rear perspective view of the upper movable base of FIG. 15;

FIG. 18 a front perspective view of a system for positioning a needle for treatment of the prostate of a patient according to a second embodiment;

FIG. 19 is a rear elevation view of the system for positioning a needle for treatment of the prostate of a patient of FIG. 18;

FIG. 20 is a block diagram of a control system for the system for positioning a needle for treatment of the prostate of a patient of FIGS. 7 and 18;

FIG. 21 is a screenshot of an operator console in the control system of FIG. 20.

Like numerals represent like features on the various drawings.

### DETAILED DESCRIPTION

Various aspects of the present disclosure generally address one or more of the inconveniences caused by the use of conventional, bulky equipment for manipulation of needles used by clinicians for diagnosis, or treatment of the prostate. The disclosed technology is also applicable to other medical uses and to other uses that require precise positioning of tools.

A tool manipulator as disclosed herein includes a base, a caliper, a tool holder and an actuator. The base is adapted to be mounted to an operation table. The caliper is supported by the base and the tool holder is mounted on the caliper. When the base is integrated in the operation table, the tool manipulator occupies limited space between its operator (usually a clinician such as a surgeon) and a patient because the actuator is located below a patient supporting surface of the operation table. The actuator is therefore out of sight of the operator who is unencumbered by bulky mechanisms of conventional equipment. The actuator can move the tool in at least three degrees of freedom. In a particular embodiment, the actuator can move the base in two degrees of freedom and also move the caliper and tool holder in three additional degrees of freedom, providing the operator with fine adjustment of a tool position over five degrees of freedom. The tool manipulator and the operation table can be made part of a system for positioning a needle for diagnosis or treatment of the prostate of a patient. The system also comprises controller connected to a power source for providing positioning commands to the actuator.

While the foregoing discussion expresses use of the tool manipulator and of the system for positioning a needle in the context of diagnosis or treatment of the prostate, the present disclosure is not limited to such uses. The tool manipulator or its variants may be put to use for manipulation of needles or similar thin and elongated devices in various medical uses as well as in non-medical uses requiring precise tool positioning. Without limitation, the system for positioning a needle or its variants may be used for gynecological applications, for example for interventions in the cervix.

The following terminology is used throughout the present disclosure:
Tool manipulator: an apparatus for holding and directing a tool, for example a needle for medical use.
Needle: any one of various types of needles usable in the medical domain; in the case of needles used for diagnosis or treatment of the prostate, these may include without limitation needles adapted for biopsy, brachytherapy, drug delivery and cryotherapy.
Operation table: a support on which a patient may lie for undergoing a medical procedure.
Patient supporting surface of an operation table: the actual surface on which the patient rests.
Actuator: a mechanical device for controlling or moving something.
Pneumatic actuator: a type of actuator using pneumatic (e.g. air) pressure to control or move something.
Positioning command: a control signal intended to move the position of an actuator, for example pneumatic pressure.
Degrees of freedom: a number of independent motions of a mechanism.
Pneumatic brake: a mechanical device using pneumatic (e.g. air) pressure to prevent something from moving.
Blocking command: a control, for example pneumatic pressure, intended to prevent movement of a mechanism.
Optical detector: a sensor of light for determining the position of an object.
Perineum conditioner: a small frame including a puncturable section, configured for being placed against the perineum of a patient.
Controller: a processor, a computer, a combination of processors and/or computers, possibly including a memory, an interface, and similar components, the controller may be hard-wired for carrying a function or may comprise programmable code for carrying a function.
Power source: a device providing power to an actuator as instructed by a controller.
Pneumatic source: a type of power source providing pneumatic pressure to pneumatic actuators and to pneumatic brakes as instructed by a controller.
Operator console: a controller or computer, a display and an input interface that together allow an operator to control a system.
Hip positioner: a component of an operation table for raising and lowering the hips of a patient and/or for modifying an angle of the hips of a patient.
Footrest: a component of an operation table for resting and positioning a foot of a patient.
Step-by-step: movement of a device component in minute steps.
Medical imaging system: a system supporting one of various techniques for rendering a visual representation of the interior of a body or a part thereof.

The disclosed tool manipulator can be used for guiding various tools, for example drills, needles, screwdrivers, blades, awls, and the like. The tool manipulator is generally usable in applications that involve delicate positioning of a tool. Without limitation, such applications include medical applications, more particularly surgical applications. The following description and the drawings provide non-limiting application examples for use in diagnostic and treatment of illnesses related to the prostate.

### First Embodiment of a Needle Manipulator

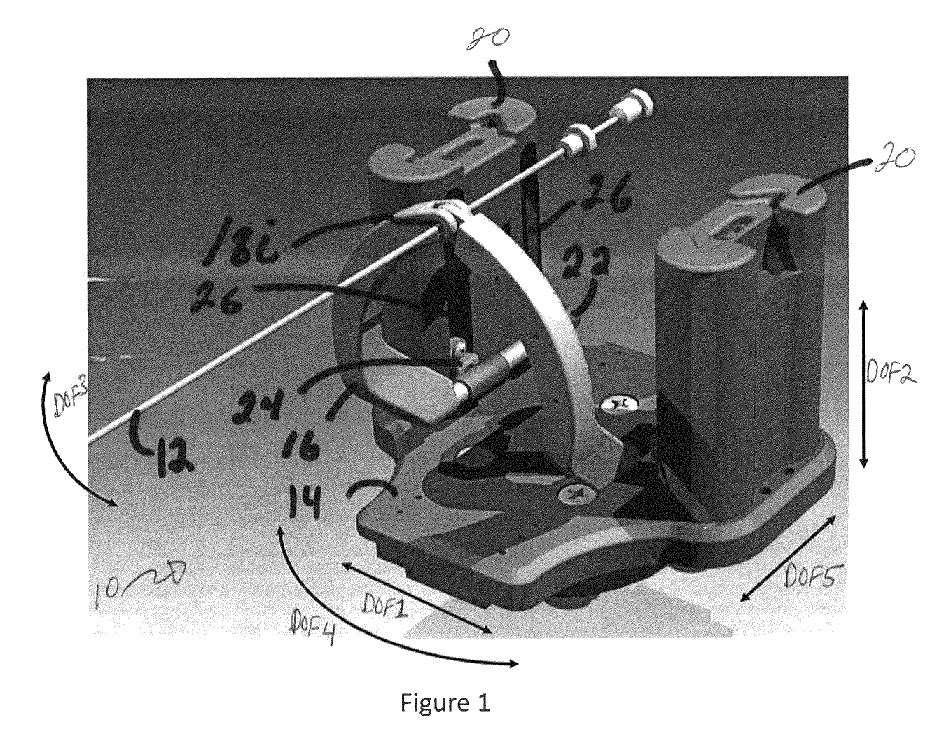

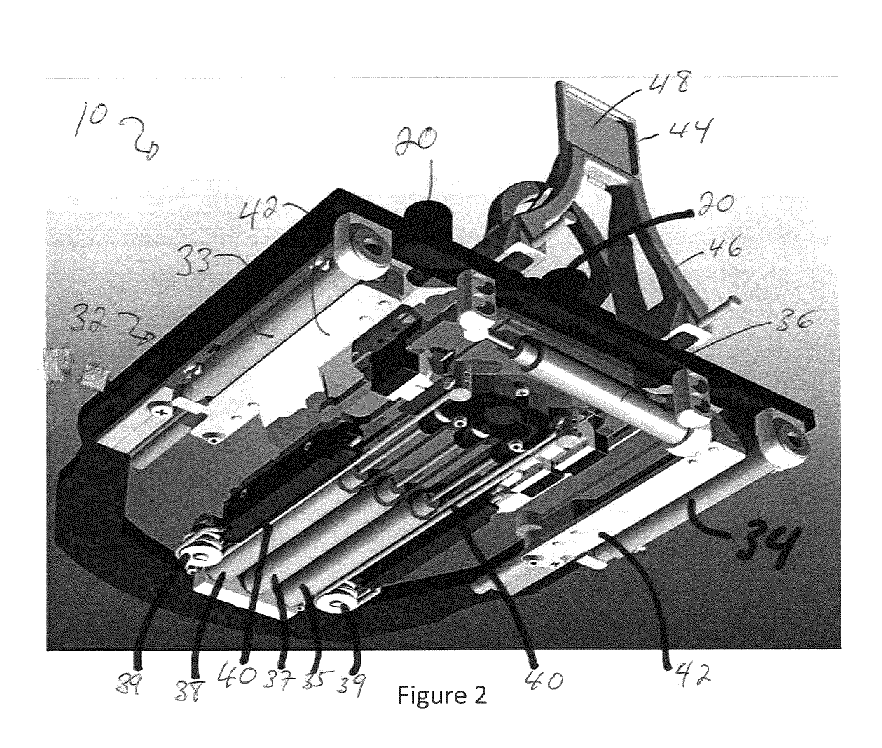

For example, FIG. 1 is a top perspective view of a needle manipulator according to a first embodiment. FIG. 2 is a bottom perspective view of the needle manipulator of FIG. 1. Referring at once to FIGS. 1 and 2, which show various components of a needle manipulator 10 adapted to hold a needle 12 used for diagnosis or treatment of the prostate, or for other medical uses. The needle manipulator 10 comprises a base 14, a pair of towers 20 mounted on the base 14, a caliper 16 supported by the towers 20, a needle holder 18i, which is integrated to the caliper 16, and an actuator. The base 14 is configured to be mounted on an operation table (shown on later Figures). When the needle manipulator 10 is mounted on the operation table, the actuator is located below a patient supporting surface (shown on later Figures) of the operation table.

The actuator is configured to move the needle 12 in up to five (5) degrees of freedom. The caliper 16 is attached to a pair of parallel stems 22, each stem 22 being supported by a pair of brackets 24 mounted within parallel, vertical and elongated slots 26 of the towers 20. Moving up and down two (2) brackets 24 located within slots 26 of a same tower 20 rotates the caliper 16, translating the needle 12 to the left or to the right in a first degree of freedom (DOF1). Simultaneously moving all four (4) brackets 24 up and down along their respective slots 26 moves the stems 22, the caliper 16, the needle holder 18i and the needle 12 vertically along a second degree of freedom (DOF2). Moving up or down one bracket 24 per tower 20, either including those closer to the caliper 16 or those farther from the caliper 16, modifies a pitch of the caliper 16 in relation to the patient supporting surface, changing a vertical angle of the needle 12 in a third degree of freedom (DOF3). Optionally, rotating the base 14 horizontally about an axis (not shown) perpendicular to a plane of the operation table moves the needle 12 in a fourth degree of freedom (DOF4). The actuator may further be configured to move the base 14 in a fifth degree of freedom (DOF5), horizontally along a length of the operation table (from front to back). These movements of the base 14 and of the caliper 16 (including the needle holder 18i) effectively provide for moving the needle 12 in at least three (3) and up to five (5) degrees of freedom. Though motion of the needle manipulator 10 can be actuated independently over each of the five (5) degrees of freedom, provision of compounded commands for simultaneously moving the needle 12 over a plurality of degrees of freedom is also contemplated.

In the embodiment shown on FIG. 2, the actuator is a pneumatic actuator 32 and includes six (6) low friction pneumatic cylinders. These cylinders contribute to move the needle 12 over five (5) degrees of freedom. The pneumatic actuator 32 receives positioning commands from a controller (shown on later Figures) for moving the base 14 in two (2) degrees of freedom and for moving the caliper 16 in three (3) additional degrees of freedom. Some cylinders directly actuate the base 14 of the needle manipulator 10 while some other cylinders are connected via pulleys 39 and cables 40 to the brackets 24 connected to the stems 22 and to the caliper 16. Not all details of pulleys, cables and other elements of the pneumatic actuator 32 are shown in order to simplify the illustration.

In more details, pneumatic cylinders 35, 37 and 38 are operably connected to the four (4) brackets 24 via the pulleys 39 and the cables 40. Actuation of the pneumatic cylinders 37 and 38 contributes to moving the caliper 16 in the first degree of freedom (DOF1), rotating the caliper 16 to move (i.e. translate) the needle 12 to the left or to the right. The pneumatic cylinders 35 and 38 are actuated to move the four (4) brackets 24 and the caliper 16 in the second degree of freedom (DOF2), vertically in relation to the base 14. A third degree of freedom (DOF3) is applied by actuation of the pneumatic cylinders 35 and 37 contributes to modifying a pitch of the caliper 16 in relation to the base 14, modifying a vertical angle of the needle 12. Optionally, a pneumatic cylinder 36 contributes to rotate the base 14 horizontally in the fourth degree of freedom (DOF4) and actuation of pneumatic cylinders 33 and 34 contribute to moving the base 14 in the fifth degree of freedom (DOF5), horizontally along a length of the operation table (from front to back). Operation of the needle manipulator 10 using these five (5) degrees of freedom allow to finely define a position and an insertion trajectory (or aim) of the needle 12 for insertion in the perineum of a patient. Actual longitudinal motion of the needle 12 for insertion is performed manually by a clinician.

In a variant, a single cylinder may be used for moving the base 14 in the fifth degree of freedom (DOF5), horizontally along the length of the operation table. Such a cylinder may for example be centrally located underneath a plane that includes the cylinders 35, 36, 37 and 38.

A variant of the pneumatic actuator 32 may comprise pneumatic muscles (not shown) instead of pneumatic cylinders. Use of non-pneumatic actuators, including for example step-by step motors (not shown), is also contemplated.

FIG. 2 also shows a pair of optical detectors 42 that provide a position of the needle 12 mounted to the needle holder 18. The optical detectors 42 as shown are located underneath the base 14 and track movements of the components of the pneumatic actuator 32 over the five (5) degrees of freedom. The actual position and insertion trajectory of the needle 12 are calculated based on readings of the optical detectors 42, accounting for the configuration and architecture of the needle manipulator 10. Use of an optical detector located on or above the base 14 for direct detection of the position and aim of the needle 12 is also contemplated.

FIG. 2 further shows a perineum conditioner 44 supported on the operation table by a bracket 46. The bracket 46 and the perineum conditioner 44 can be manually moved forward or backward by the clinician over a short range, for example within a 3 or 4 cm course, until it is positioned against the perineum of the patient. A button or similar control (not shown) may be used to lock the perineum conditioner 44 in place. Unlike a prostate template of a conventional needle guide used for prostate treatment, the perineum conditioner 44 does not comprise preformed holes for guiding a needle. Instead the perineum conditioner 44 consists of a small frame including a puncturable section 48. The section 48 may be made of silicon or similar transparent materials. In use, the perineum conditioner 44 is placed against the perineum of the patient before insertion of the needle 12, usually before adjustment of the position and trajectory of the needle 12. The needle 12 pierces the section 48 upon insertion in the perineum. This helps reducing flexing of the needle 12 upon insertion in the perineum and helps maintaining the needle 12 in place once inserted in the perineum. The needle manipulator 10 can be used to successively insert more than one needle 12 in the course of a single procedure and the section 48 can maintain several needles in place. Fine adjustment over the five (5) degrees of freedom allows inserting a needle between two (2) previously installed needles, preventing collision between these needles. The perineum conditioner 44 with the section 48 can be replaced after each procedure.

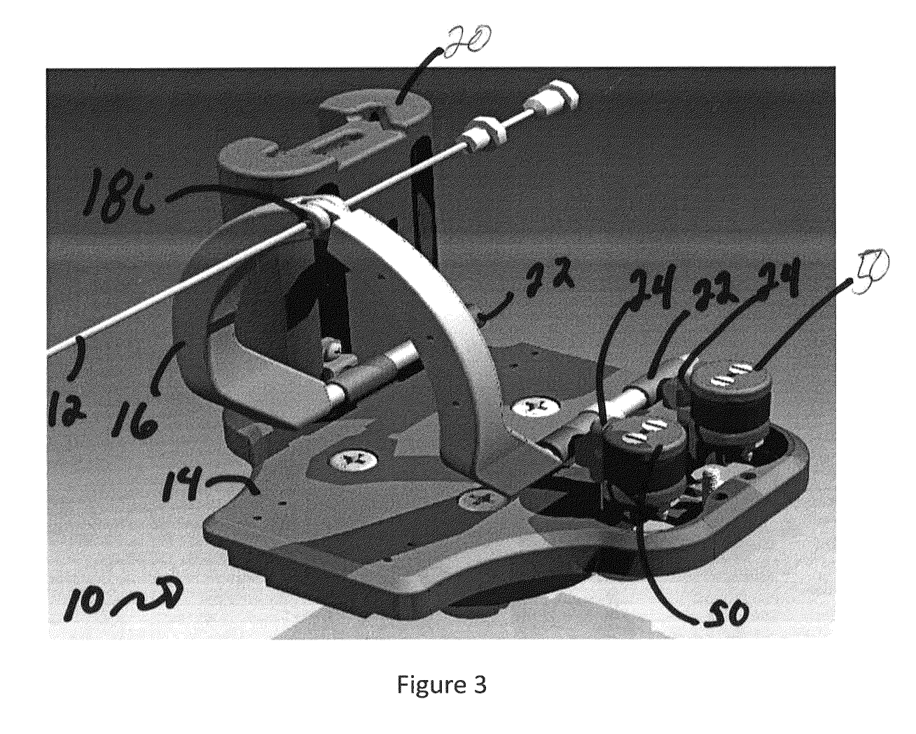

FIG. 3 is a perspective view of the needle manipulator of FIG. 1 showing a detail of a pneumatic brake. One of the towers 20 is removed to show pneumatic brakes 50. One or more pneumatic brakes are mounted on the base 14, under at least one or both of the towers 20. The pneumatic brakes 50 are connected to the pneumatic actuator 32 and/or to the brackets 24. The pneumatic brakes 50 are used to prevent movements of the base 14 and of the caliper 16 when receiving a blocking command from the controller. The pneumatic brakes 50 may also prevent movements of the bracket 46 and of the perineum conditioner 44.

### Second Embodiment of a Needle Manipulator

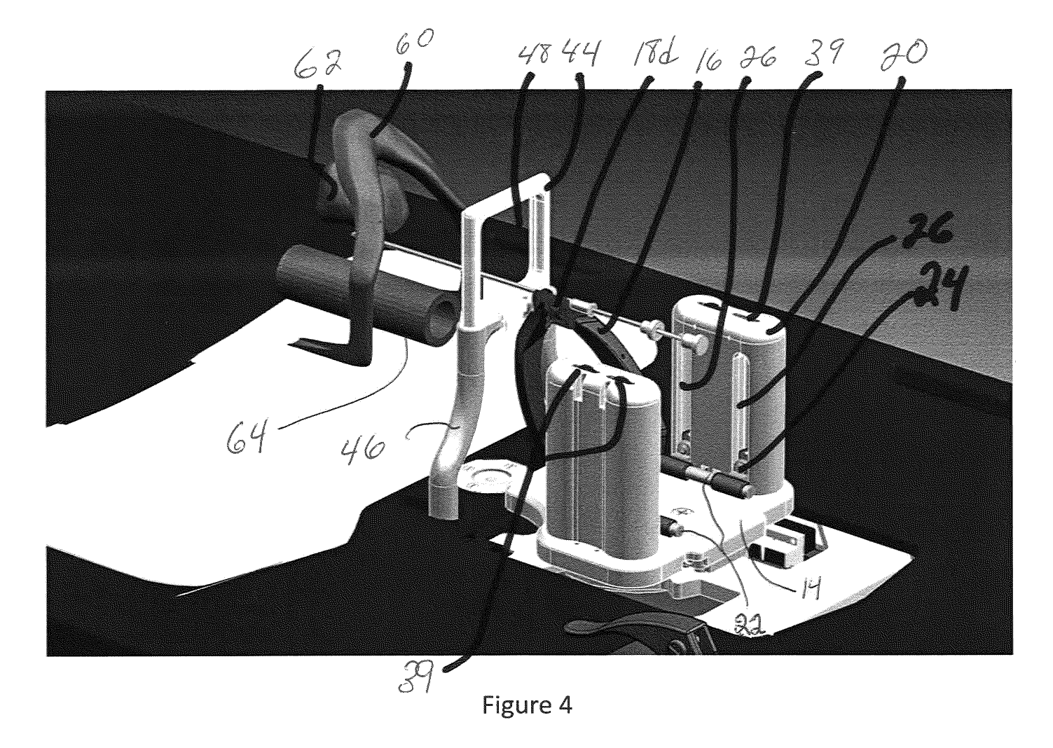

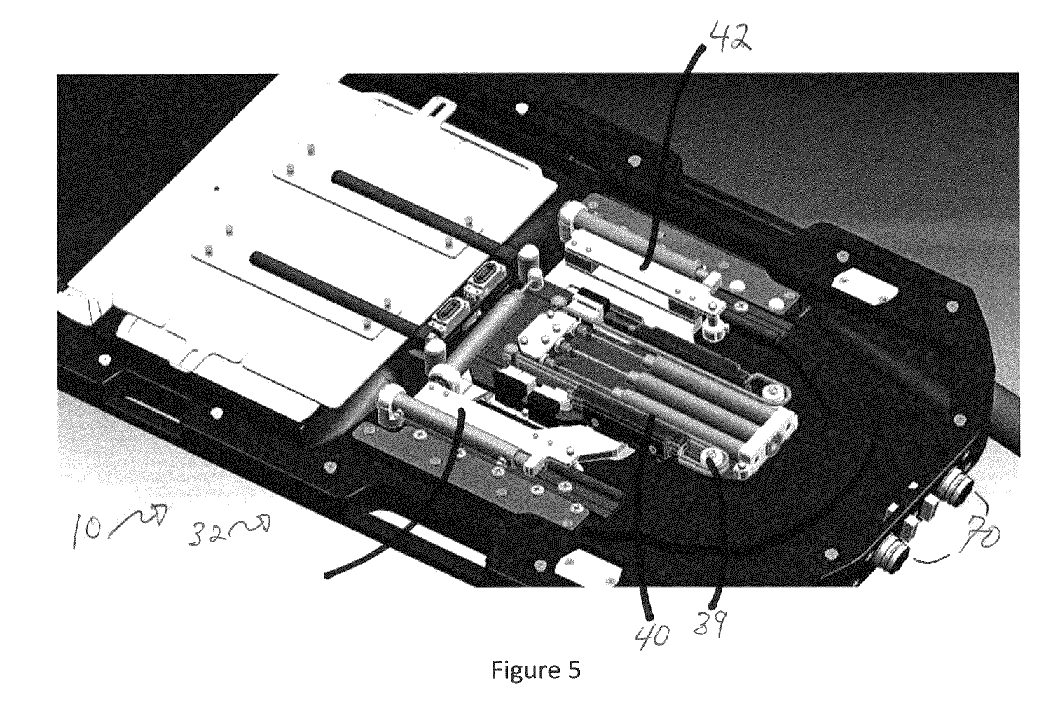

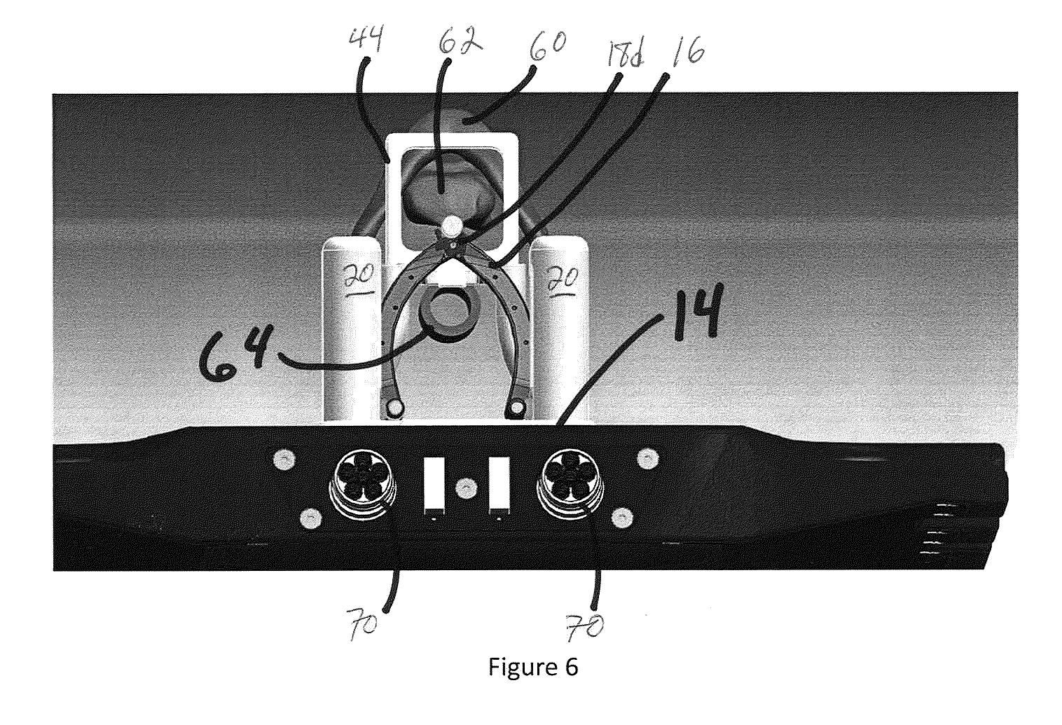

FIG. 4 is a top perspective view of a needle manipulator according to a second embodiment. FIG. 5 is a bottom perspective view of the needle manipulator of FIG. 4. FIG. 6 is a rear elevation view of the needle manipulator of FIG. 4. The first and second embodiments of the needle manipulator 10 are similar. The following description therefore highlights additional features illustrated on FIGS. 4 to 6.

As shown on FIGS. 4 and 6, the integrated needle holder 18i of earlier Figures is replaced by a detachable needle holder 18d. The needle holder 18d is fixedly attached to the caliper 16 by a clip (not explicitly shown) and can be detached after use. The needle holder 18d is configured for easy detachment of a needle 12 having been inserted in the perineum, so to facilitate mounting of another needle 12, facilitating procedures that require insertion of a plurality of needles. Without limitation, the needle holder 18d can accommodate needles of 12 to 20 gauge. The needle holder 18d can be replaced after each procedure.

FIGS. 4 and 6 show the pubic arch 60 and the prostate 62 of the patient. An endorectal coil 64 used for magnetic resonance imaging (MRI) (or an ultrasound probe) is also schematically shown. As visible on FIG. 6, the caliper 16 is shaped to provide the clinician free access for insertion of the endorectal coil 64. Also shown on the various Figures are pneumatic connectors 70 mounted to the base 14 and connected to the pneumatic cylinders 33-38.

The above described elements of the needle manipulator 10 may be constructed using a variety of materials. In some embodiments, the needle manipulator 10 can be constructed using nonmagnetic and dielectric materials for MRI compatibility. Some commercially available pneumatic actuators have good MRI compatibility. In a variant, a few fiducial markers (not shown) may be inserted in the base 14 and in the caliper 16. Detection of the position of the fiducial markers by MRI facilitates a determination of the position and trajectory of the needle 12 in relation to the patient and, specifically, in relation to his prostate.

### First Embodiment of a System for Positioning a Needle for Treatment of the Prostate of a Patient

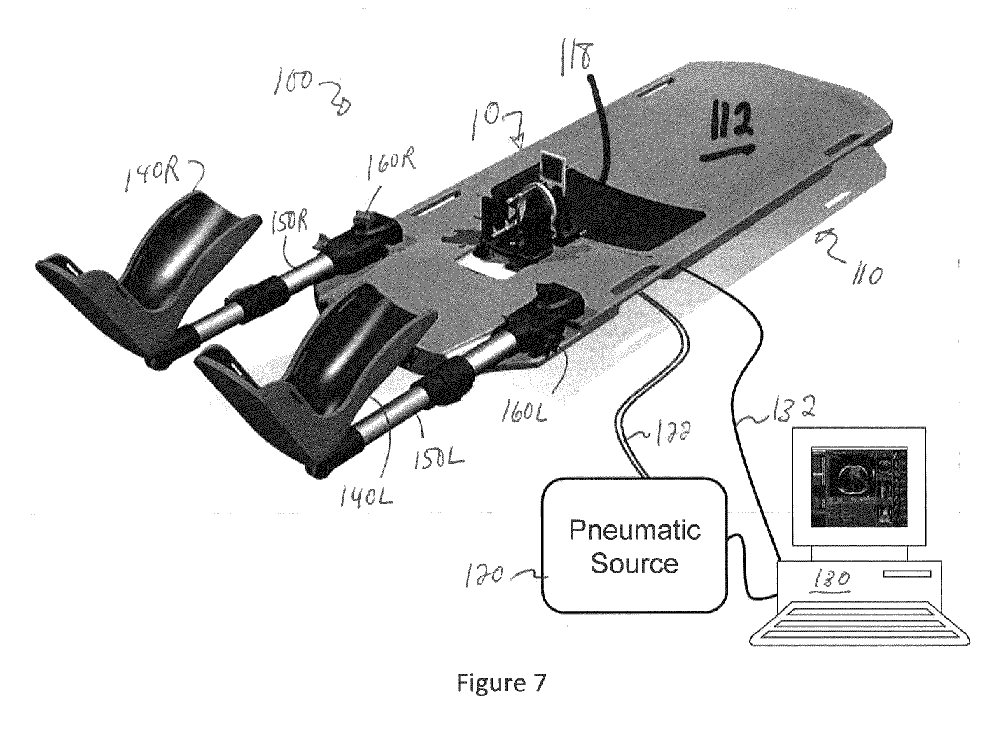

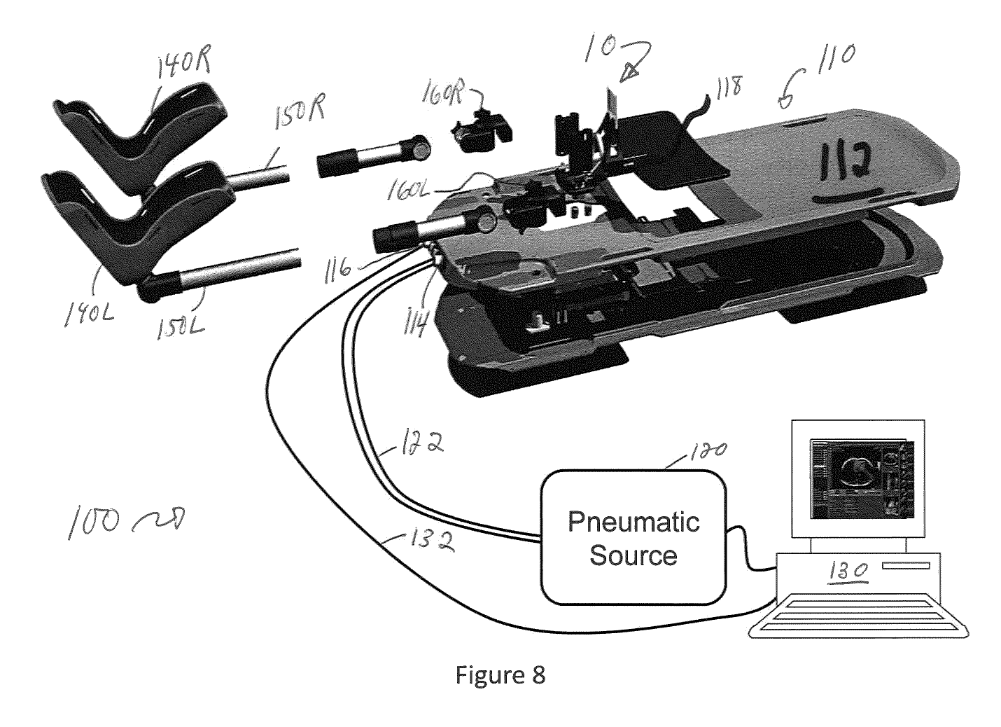

FIG. 7 is a perspective view of a system for positioning a needle for treatment of the prostate of a patient according to a first embodiment. The system of FIG. 7 incorporates the needle manipulator of FIGS. 1-3 or the needle manipulator of FIGS. 4-6. FIG. 8 is an exploded view of the system of FIG. 7. These Figures clearly show the limited bulk of the needle manipulator 10 in relation to the positions of the patient and of the clinician. Referring at once to FIGS. 7 and 8, a system 100 for positioning a needle for diagnosis or treatment of the prostate of a subject includes the needle manipulator 10, an operation table 110, a pneumatic source 120 and a controller 130. The needle manipulator 10 is integrated in the operation table 110, the base 14 being substantially at the level of a patient supporting surface 112, the pneumatic actuator 32 being at a lower level compared to the patient supporting surface 112. Patient restraints (not shown) may be integrated to the operation table 110.

The controller 130 is connected to the pneumatic source 120 and controls provision of the positioning commands from the pneumatic source 120 to the pneumatic actuator 32 as well as provision of the blocking commands from the pneumatic source 120 to the pneumatic brakes 50. For compatibility issues with medical imaging technologies, such as for example MRI, the controller 130 may be located outside of a room where the operation table 110 is installed. The pneumatic source 120 is connected to the pneumatic actuator 32 and to the pneumatic brakes 50 via a pneumatic connection 122 routed through a pneumatic connector 114 of the operation table 110. The pneumatic connection 122 may include a plurality of distinct lines and may be connected to the operation table 110 via a plurality of connectors. Only one is shown for simplicity, without limiting the present disclosure. The pneumatic source 120 may include a compressor, a regulator, and an assortment of pneumatic valves (not shown).

An optical fiber connection 132 connects the controller 130 to the needle manipulator 10 through an optical connector 116 of the operation table 110. Positioning information detected by the optical detectors 42 of the needle manipulator 10 are provided to the controller 130 via the optical fiber connection 132. The controller 130 uses this positioning information, which relates to internal movements within the pneumatic actuator 32, to calculate the actual position and trajectory of the needle 12.

A pneumatic hip positioner 118 is integrated within the patient supporting surface 112 of the operation table 110. The pneumatic hip positioner 118 is used to adjust a height and/or an angle of the hips of a patient lying on the supporting surface 112 in relation to the needle manipulator 10. A balloon (not shown) placed underneath a top part of the pneumatic hip positioner 118 is inflated or deflated to raise or lower the hips of the patient. The pneumatic hip positioner 118 is also connected to the pneumatic source 120 via the pneumatic connection 122 and the pneumatic connector 114. The controller 130 gives commands to the pneumatic source 120 to control operation of the pneumatic hip positioner 118. Addition of a head positioner (not shown) to the operation table 110 for adjusting a height and/or an angle of the head of the patient is also contemplated.

Various components of the needle manipulator 10 as well as the pneumatic hip positioner 118 are connected via pneumatic and optical cables (not shown) that run underneath the patient supporting surface 112 up to the pneumatic connector 114 and optical the connector 116. Though FIG. 8 shows the pneumatic connector 114 and optical the connector 116 being mounted at one end of the operation table 110, between the legs of the patient, they may be mounted at other places around the perimeter of the operation table 110, for example at the opposite extremity, close to the head of the patient.

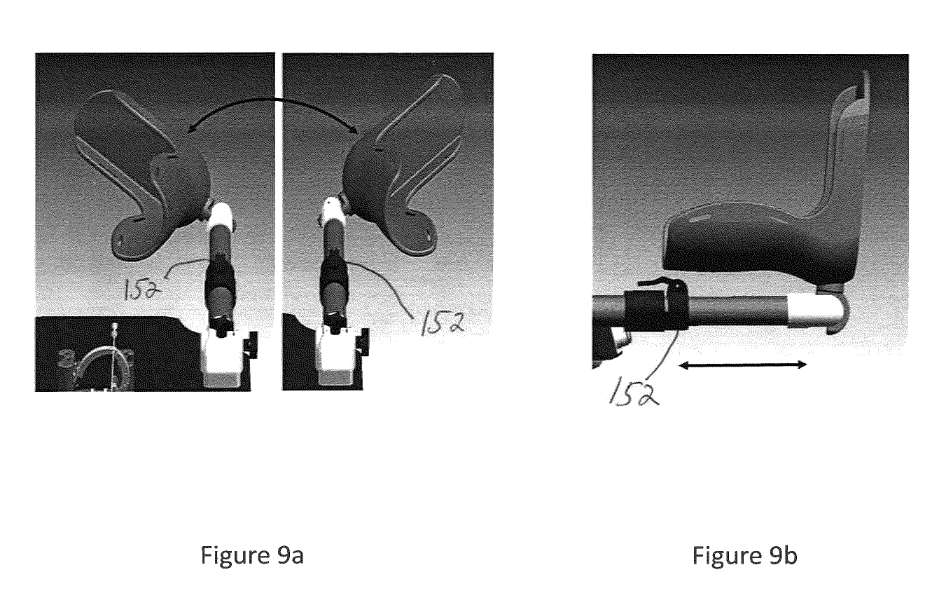

The system 100 also includes a pair of footrests 140L and 140R, attached to extensible legs 150L and 150R that are mounted to the operation table 110 via adjustable supports 160L and 160R. FIGS. 9a-9d are detailed views of footrests of the system of FIG. 7, showing their adjustability over four (4) degrees of freedom. The four degrees of freedom of the footrests 140L, 140R include:
Rotational adjustment of the footrests 140L, 140R by un locking and locking again latches 152 (one on each side) for extension of the legs 150L, 150R (FIG. 9a);
Lengthwise adjustment of the footrests 140L, 140R also by operation of the latches 152 for extension of the legs 150L, 150R (FIG. 9b);
Adjustment of a width between the footrests 140L, 140R by operation of knobs 164 of the supports 160L, 160R (FIG. 9c); and
Up and down adjustment of the footrests 140L, 140R by operation of knobs 162 of the supports 160L, 160R (FIG. 9d).

Use of controllable pneumatic adjustors (not shown) to modify a position of the footrests 140L, 140R is also contemplated.

### Third Embodiment of a Needle Manipulator

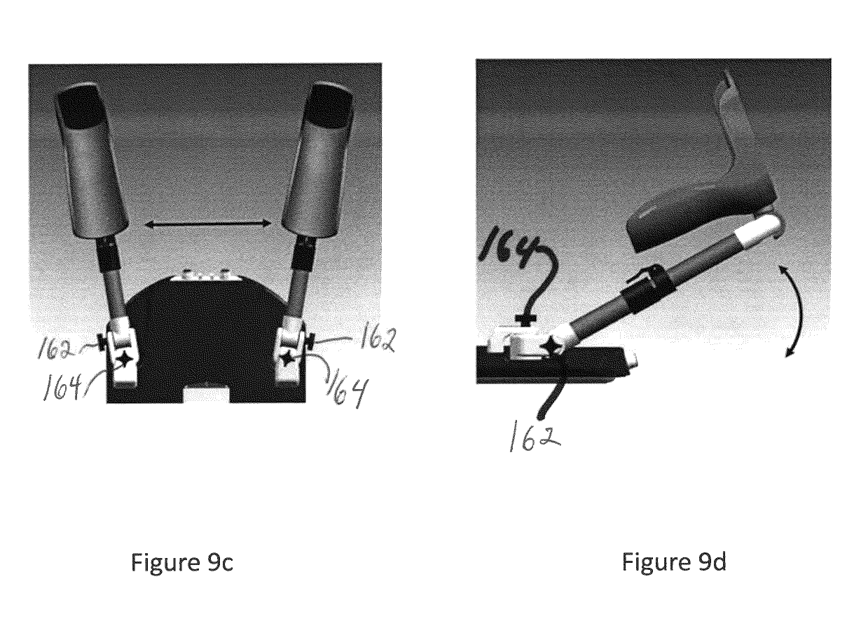

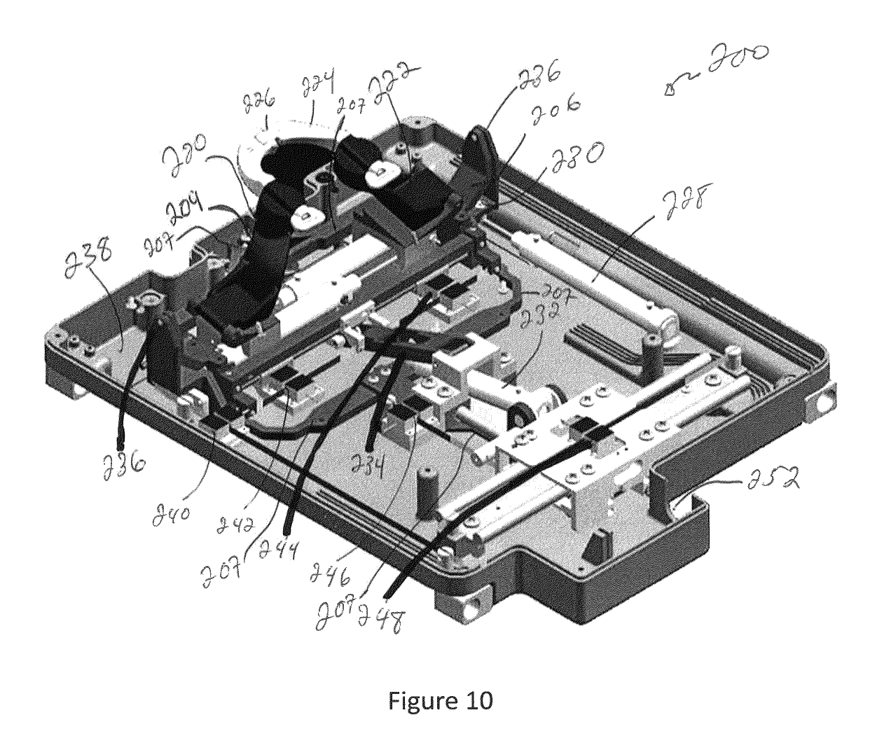

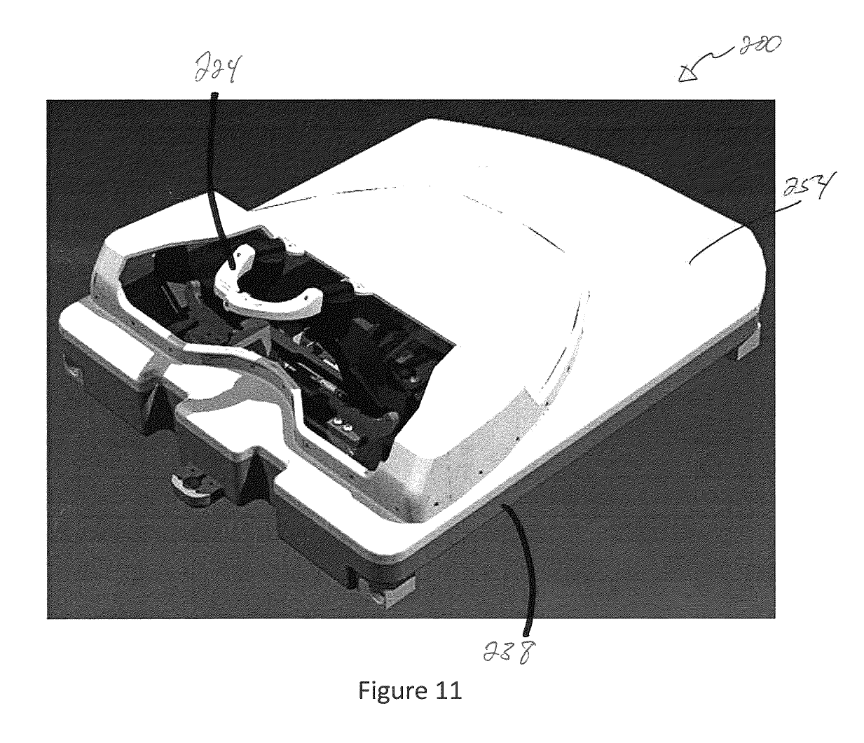

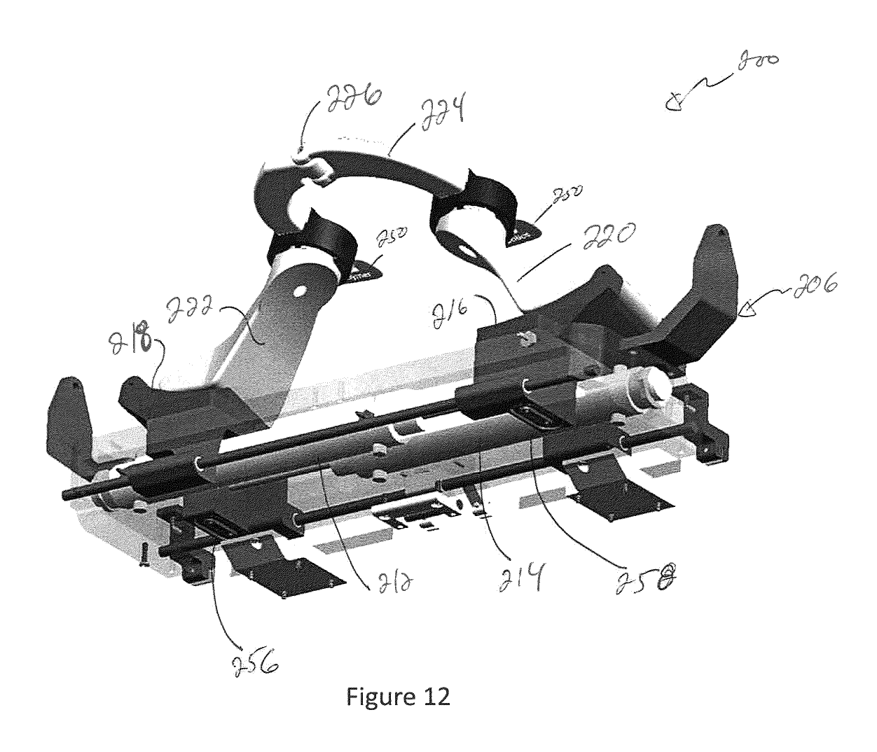

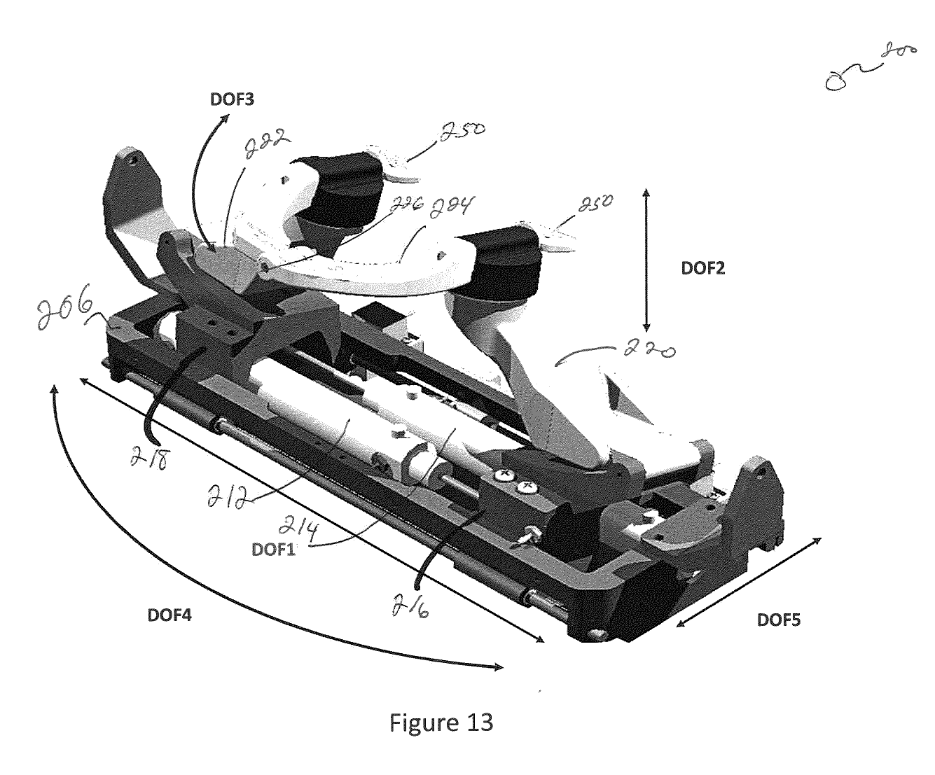

FIG. 10 is a rear perspective view of a needle manipulator, shown without a cover, according to a third embodiment. FIG. 11 is a front perspective view of the needle manipulator of FIG. 10, shown with a cover. FIG. 12 is a bottom perspective view a upper movable base and of a needle support of the needle manipulator of FIG. 10. FIG. 13 is a top perspective view of the upper movable base and of the needle support of FIG. 12. FIG. 14 is a bottom plan view of the needle manipulator of FIG. 10, shown without a cover. Referring at once to FIGS. 10-14, a needle manipulator 200 includes a caliper 224 supported by a pair of arms 220, 222, the caliper 224 and the arms 220, 222 forming a needle support. The needle support is mounted to a movable base that includes a upper movable base 206 that is itself mounted to a lower movable base 207. The lower movable base 207 can pivot horizontally about a pivot point 209 of a platform 238 that supports the various components of the needle manipulator 200. A pair of cylinders 208 coupled to the upper movable base 206 via a pair of elongated rods 210 allow the movable base to move longitudinally along the same degree of freedom DOF5 as in the case of the needle manipulator 10 of earlier Figures. Within the upper movable base 206, a pair of cylinders 212 and 214 allows the needle support to move sideways, from left to right, in the first degree of freedom DOF1 of earlier Figures. In more details, the cylinders 212 and 214 each includes a piston connected to a cradle 216 and 218, respectively. Each cradle 216, 2218 supports a respective arm 220 and 222 that in turn support the caliper 224, which has a needle holder 226. Moving the two cradles 216, 218 closer at once raises the arms 220 and 222, in turn raising the caliper 224 along a vertical degree of freedom DOF2. Likewise, moving the two cradles 216, 218 apart lowers the arms 220 and 222, in turn lowering the caliper 224.

Another cylinder 228 has a piston connected to one end 230 of the lower movable base 207 and allows rotating the lower movable base 207 and all elements mounted thereon about a degree of freedom DOF4, about a vertical axis, about a degree of freedom DOF3. The upper movable base 206 is pivotably mounted to brackets 236 that extend upright from the lower movable base 207 and can pivot about a horizontal axis. Another cylinder 232 is mounted on the lower movable base 207 to follow its movement about the degree of freedom DOF4. The cylinder 232 is connected to the upper movable base 206 via an angled lever 234. Actuation of the cylinder 232 allows rotating the upper movable base 206 and all components mounted thereon about a degree of freedom DOF3.

Rubber membranes 256 and 258 act as pneumatic brakes to provide braking functions for the cylinders 212 and 214. Similar membranes (not shown) may provide braking functions for the other cylinders 208, 228 and 232.

Optical detectors 240, 242, 244, 246 and 248 are positioned on the platform 238 of the needle manipulator 200 and provide positioning information of the needle manipulator 200 about degrees of freedom DOF5, DOF2, DOF1, DOF3 and DOF4, respectively. One or more openings such as 252 may be provided on the platform 238 allowing the passage of conduits such as optical fibers or electrical wires (not shown) connecting the optical detectors 240, 242, 244, 246 and 248 to an external controller (shown on later Figures) and/or pneumatic conduits connected to the various cylinders.

As an optional feature, thumb screws 250 may be used to easily and replaceably mount the caliper 224 on the arms 220 and 222.

A cover 254 generally hides and protects most components of the needle manipulator 200.

### Fourth Embodiment of a Needle Manipulator

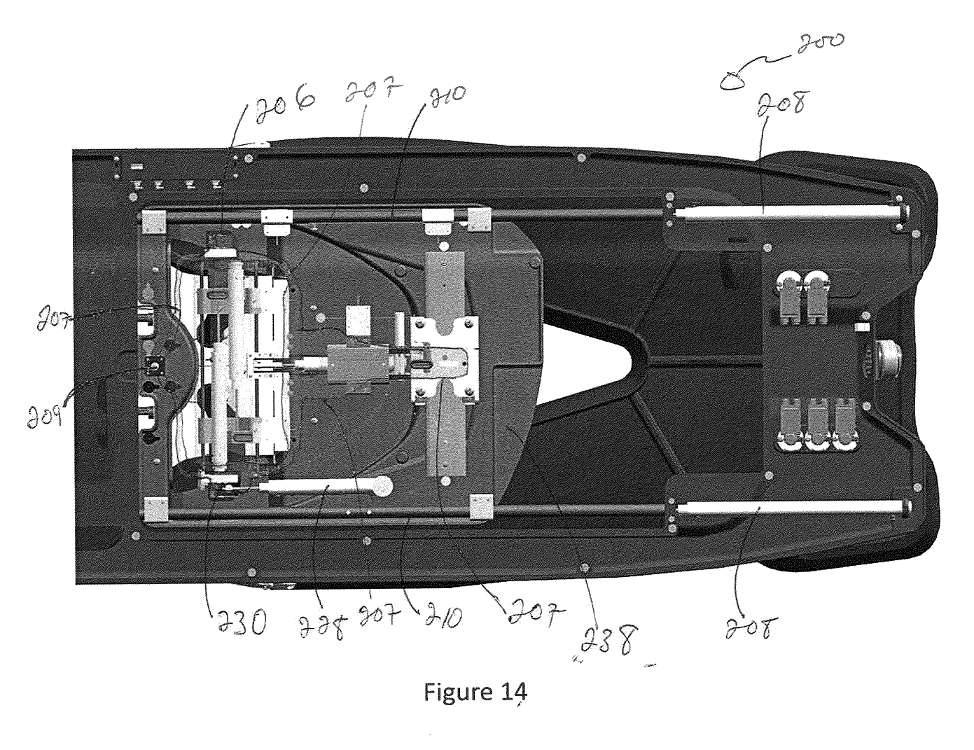

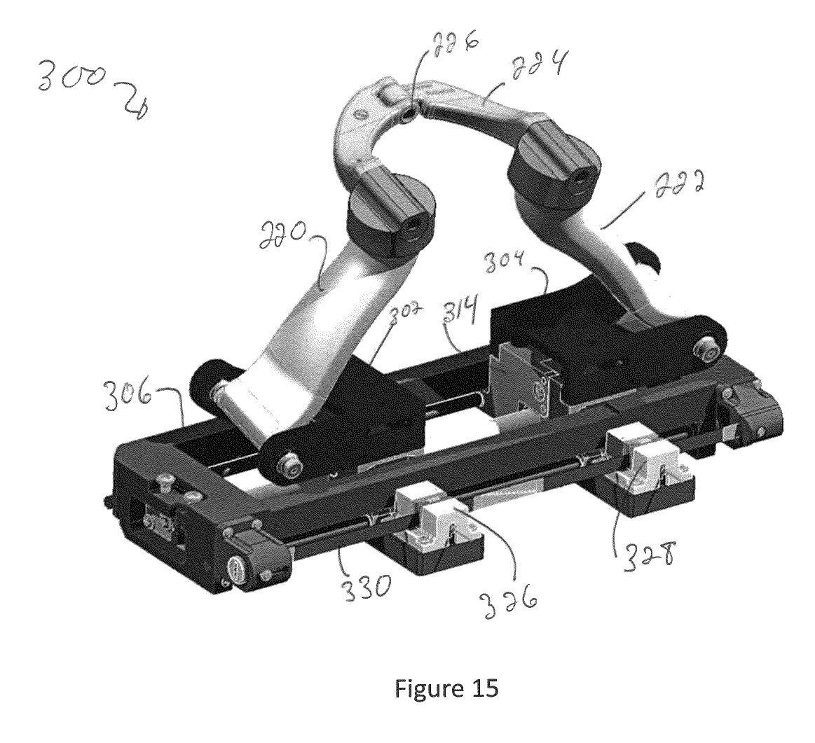

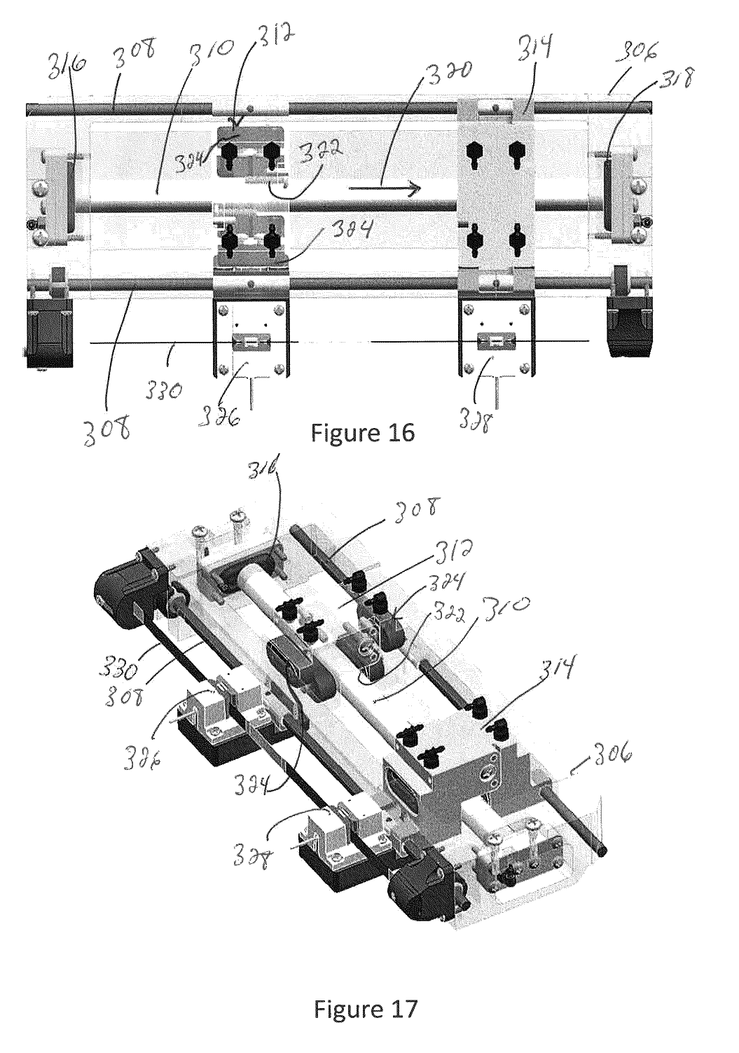

FIG. 15 is a rear perspective view of a upper movable base and of a needle support of a fourth embodiment of a needle manipulator. FIG. 16 is a top view of the upper movable base of FIG. 15. FIG. 17 is rear perspective view of the upper movable base of FIG. 15. FIGS. 15-17 collectively show differences between this fourth embodiment and the third embodiment of FIGS. 10-14. These embodiments of the needle manipulator are similar and only their differences are described in the next few paragraphs.

In a needle manipulator 300, the caliper 224 is still supported by the arms 220, 222, which are mounted to a modified upper movable base 306 via modified cradles 302 and 304. The upper movable base 306 is mounted to the same lower movable base 207 described hereinabove. The cradles 302 and 304 have the same function as in the case of the cradles 216 and 218, but are not connected to pneumatic cylinders. Instead, the cradles 216 and 218 are connected to a step-by-step pneumatic system according to an aspect of the present disclosure. The upper movable base 306 includes a pair of transversal rails 308. An oscillating rod 310 is mounted between the rails 308, being parallel to the rails 308. A pair of chariots 312 and 314 is supported by the rails 308, riding on the oscillation rod 310. The upper movable base 306 and the chariot 312 are shown in transparency in FIGS. 16 and 17 in order to provide a better view of the step-by-step pneumatic system.

The oscillating rod 310 is mounted to the upper movable base 306 between a pair of pulsating pneumatic end membranes 316 and 318. A length of the oscillating rod 310 is reduced by a small gap compared to a space available between the end membranes 316 and 318 when no pneumatic pressure is applied to the end membranes 316 and 318. Without limitation, the small gap may for example be in a range of 0.5 mm to 1.0 mm.

Applying pressure on the end membrane 316, usually in the absence of pressure on the end membrane 318, forces the oscillating rod 310 to move toward the other end of the upper movable base 306, in the direction of the arrow 320. Likewise, applying pressure on the end membrane 318 in the absence of pressure on the end membrane 316 forces the oscillating rod 310 to move in the opposite direction.

Each of the chariots 312 and 314 includes pneumatic membranes. Considering for example the chariot 312, it includes a coupling membrane 322 for coupling the chariot 312 to the oscillating rod 310, and at least one braking membrane 324 (two such braking membranes 324 are shown) for coupling the chariot 312 to at least one of the rails 308. The chariot 312 is moved in the direction of the arrow 320 by following a few steps, under the control of a pneumatic control system (shown on other Figures):
Step 1: Pressure is applied on the coupling membrane 322 to solidarize the chariot 312 to the oscillating rod 310.
Step 2: Pressure is applied on the end membrane 316, causing the oscillating rod 310 and the chariot 312 to move in the direction of the arrow 320.
Step 3: Pressure is applied on the braking membranes 324.
Step 4: Pressure is released on the coupling membrane 322.
Step 5: Pressure is released on the end membrane 316.
Step 6: Pressure is applied on the end membrane 318, causing the oscillating rod 310 to move in the direction opposite to the arrow 320 while the chariot 312 remains in fixed position.

The above sequence of steps may be repeated as many times as necessary until the chariot 312 reaches a desired position. Of course, execution of Step 1 will include releasing the pressure on the braking membrane 324 in order to allow further movement of the chariot 312. The chariot 312 can be moved in the opposite direction. The chariot 314 can be moved in the same manner. Both chariots 312 and 314 can be moved concurrently, for example to both move them in a same direction (degree of freedom DOF1) or in opposite directions (degree of freedom DOF2).

Some of the above described steps may be combined or otherwise concurrently executed, and the order of some of the steps may be modified. The sequence of step is detailed for clarity of the illustration of the step-by-step pneumatic system and do not limit the present disclosure.

Though not illustrated, variants of the step-by-step pneumatic system may be used to replace one or more of the other cylinders of previous Figures.

It will be appreciated that the step-by-step pneumatic system can be used for other applications, independently from its integration into the present tool manipulator. An oscillating rod can be mounted on a frame similar to the upper movable base 306, the frame supporting at least one rail parallel to the oscillating rod and supporting end membranes at each end of the oscillating rod. One or more chariots may ride on the rail and oscillating rod, each chariot having coupling and braking membranes for moving step-by-step along the oscillating rod.

A pair of optical detectors 326 and 328 is coupled to the chariots 312 and 314 and move at the same time. An encoded strip 330, for example a textile strip, is attached to extremities of the upper movable base 306. The encoded strip provides positioning information to the optical detectors 326, 328, for example having alternating dark and light colored lines along its length for decoding by the optical detectors 326, 328. The optical detectors 326, 328 provide information regarding the displacement of the caliper 224 and of the needle holder 226 along degrees of freedom DOF1 and DOF2. The optical detectors 240, 242, 244, 246 and 248 of FIG. 10 can be designed in similar fashion.

As in the case of first and second embodiments, the described elements of the needle manipulators 200 and 300 may be constructed using a variety of materials. In some embodiments, the needle manipulators 200 and 300 can be constructed using nonmagnetic and dielectric materials for MRI compatibility. Some commercially available pneumatic actuators have good MRI compatibility. In a variant, a few fiducial markers (not shown) may be inserted in the platform 238 and in the caliper 224. When used to drive a needle such as the needle 12 introduced hereinabove, detection of the position of the fiducial markers by MRI facilitates a determination of the position and trajectory of the needle 12 in relation to the patient and, specifically, in relation to his prostate.

### Second Embodiment of a System for Positioning a Needle for Treatment of the Prostate of a Patient

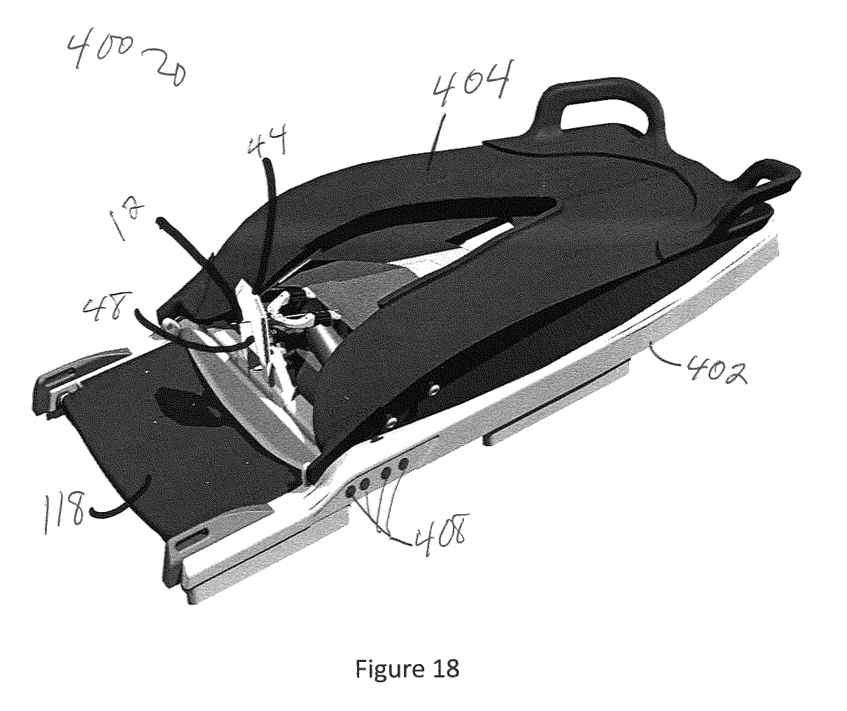

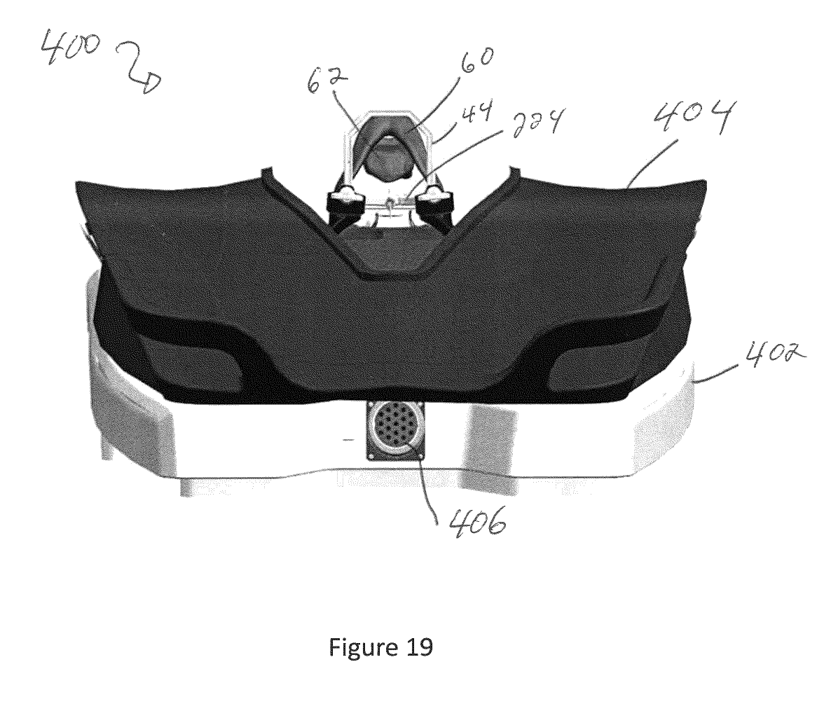

FIG. 18 a front perspective view of a system for positioning a needle for treatment of the prostate of a patient according to a second embodiment. The system incorporates the needle manipulator of FIGS. 10-14 or the needle manipulator of FIGS. 15-17. FIG. 19 is a rear elevation view of the system for positioning a needle for treatment of the prostate of a patient of FIG. 18. As shown a system 400 for positioning a needle for diagnosis or treatment of the prostate of a subject includes a frame 402 on which is mounted one of the needle manipulator 200 or 300 supporting a needle 12 piercing through the puncturable section 48 of a perineum conditioner 44. The system 400 is not limited to using the needle manipulator 200 or 300 and could also be equipped with the needle manipulator 10. The pneumatic hip positioner 118 is also shown. The footrests 140R, 140L, their supports and adjustment tools are replaced with an integral leg support 404 supported by the frame 402. The system 400 may be used in cooperation with any a patient supporting surface 112, for example a stretcher. An optical connector 406 and several pneumatic connectors 408 are mounted to the frame 402 and are operably connected to the needle manipulator 10, 200 or 300, and to the hip positioner 118. The number and position of optical, pneumatic or electric connectors may vary according to the needs of a particular application. The system 400 may be connected to the pneumatic source 120 and to the controller 130 of FIGS. 7 and 8.

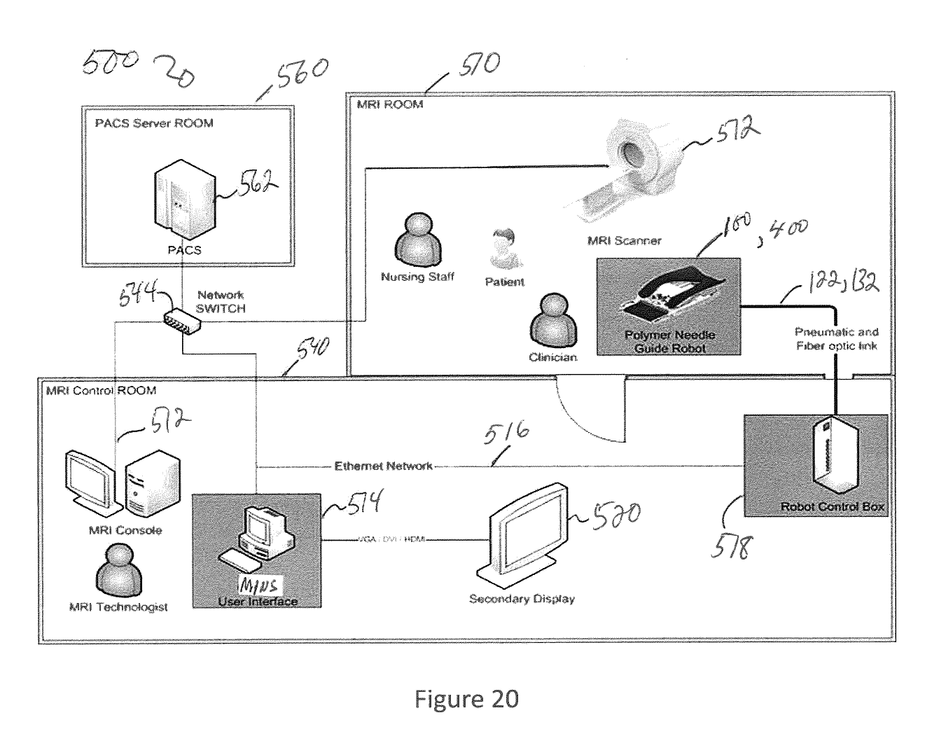

FIG. 20 is a block diagram of a control system for the system for positioning a needle for treatment of the prostate of a patient of FIGS. 7 and 18. Various elements introduced hereinabove are combined on FIG. 20 to form a network 500. The network includes elements located in an MRI room 510, in an MRI control room 540 and in a picture archiving and communication system (PACS) server room 560. As is well-known, MRI scanners such as 512 are usually placed in a first room such as 510, isolated from a control room such as 540, in order to alleviate potential electromagnetic compatibility effects between the MRI scanner 512 and computers. Because the system 100 or 400 is used while benefiting real-time imaging acquisition, the system 100 or 400 for positioning a needle for diagnosis or treatment of the prostate of a subject is installed in the MRI room 510, where the patient, a clinician and nursing staff may be present.

The system 100 or 400 and the MRI scanner 512 are both connected to equipment located in the MRI control room 540. An MRI console 540 controls the MRI scanner 512 via signals that travel through a network switch 544. Images obtained from the MRI scanner 512 may be stored in a PACS server 562 of the PACS server room 560. In the MRI control room 540, a medical imaging navigation system (MINS) user interface 514 is also connected to various elements of the network 500 via the network switch 544. The MINS user interface 514 has a direct Ethernet connection 516 to a robot control box 518. Alternatively, the MINS user interface 514 could be connected to the robot control box 518 via the network switch 544. The robot control box 518 generally includes at once the functions of the pneumatic source 120 and of the controller 130 of FIGS. 7 and 8, although some features of the controller 130 may instead by implemented as a part of the MINS user interface 514 or in a distinct computer (not shown). The robot control box 518 is connected to the system 100 or 400 via the pneumatic connection 122 and via the optical connector introduced hereinabove.

A secondary display 520 may be provided for the benefit of an additional clinician who would like to evaluate the procedure.

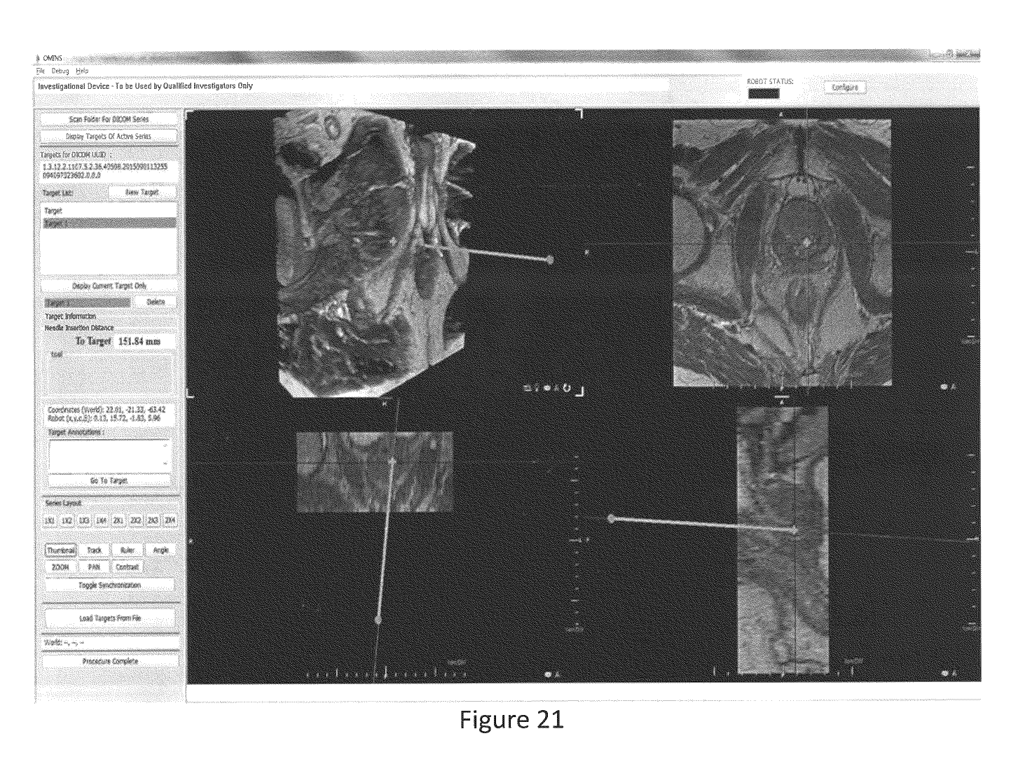

FIG. 21 is a screenshot of an operator console in the control system of FIG. 20. The operator console may be integrated in the controller 130 (as shown on FIGS. 7 and 8), in the MINS user interface 514, or in the secondary display 520 (as shown on FIG. 20), or may otherwise be communicatively coupled therewith. The operator console shows an image, for example obtained by MRI, of the region of interest of the patient, including the prostate. The position of the needle 12 may be superimposed on the image. Also shown is a variety of statuses and command icons for controlling operation of the system 100 or of the system 400. The operator console is configured to control a positioning of the patient on the operation table, a positioning of the needle manipulator, the acquisition of image information from the prostate of the patient and a verification of the placement of one or more needles in relation to the prostate of the patient.

The operator console includes navigation software to guide the clinician in operating the system 100 or the system 400. Features supported by the navigation software may include, for example:
Access to a patient record;
Communication with a picture archiving system;
Tools to enable image viewing, for example MRI in 2D and 3D;
Registration of the needle manipulator 10, 200 or 300 to a neutral (start) position;
Volume identification for segmentation of the prostate;
Target selection; and
Determination of a path to be followed by the needle 12 for a given target.

Examples of other medical imaging system that may be used as a part of, or in cooperation with the system 100 and the system 400 include, without limitation, a computerizing tomography (CT-scan) imaging system, an utrasonographic system, a positron emission tomographic system, a thermal imaging system, and a radiology system.

A workflow assisted by the operator console may for example comprise the following procedures:
1. Patient preparation (positioning, immobilization and anesthesia);
2. Return of the needle manipulator to its neutral (start) position and confirmation of the patient's position;
3. Acquisition of a high resolution image (for example by MRI) showing intended targets;
4. Target planning and confirmation of needle trajectories for reaching the targets;
5. Positioning the needle manipulator;
6. Manual insertion of the needle by the clinician;
7. Target reach confirmation by further image acquisition;
8. Insertion of additional needles, as required, by repeating operations 5 to 7; and
9. Conclusion of the procedure.

Those of ordinary skill in the art will realize that the description of the tool manipulator and of the system for positioning a needle are illustrative only and are not intended to be in any way limiting. Other embodiments will readily suggest themselves to such persons with ordinary skill in the art having the benefit of the present disclosure. Furthermore, the disclosed tool manipulator and the system for positioning a needle may be customized to offer valuable solutions to existing needs and problems related to the bulk of conventional equipment.

In the interest of clarity, not all of the routine features of the implementations of the tool manipulator and of the system for positioning a needle are shown and described. It will, of course, be appreciated that in the development of any such actual implementation of the tool manipulator and of the system for positioning a needle, numerous implementation-specific decisions may need to be made in order to achieve the developer's specific goals, such as compliance with application-, system-, regulatory-, and business-related constraints, and that these specific goals will vary from one implementation to another and from one developer to another. Moreover, it will be appreciated that a development effort might be complex and time-consuming, but would nevertheless be a routine undertaking of engineering for those of ordinary skill in the field of precision devices and systems having the benefit of the present disclosure.

In accordance with the present disclosure, the components, process steps, and/or data structures described herein may be implemented using various types of operating systems, computing platforms, network devices, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used. Where a method comprising a series of process steps is implemented by a computer or a machine and those process steps may be stored as a series of instructions readable by the machine, they may be stored on a tangible medium.

Systems and modules described herein may comprise software, firmware, hardware, or any combination(s) of software, firmware, or hardware suitable for the purposes described herein. Software and other modules may reside on servers, workstations, personal computers, computerized tablets, personal digital assistants (PDA), and other devices suitable for the purposes described herein. Software and other modules may be accessible via local memory, via a network, via a browser or other application or via other means suitable for the purposes described herein. Data structures described herein may comprise computer files, variables, programming arrays, programming structures, or any electronic information storage schemes or methods, or any combinations thereof, suitable for the purposes described herein.

Although the present disclosure has been described hereinabove by way of non-restrictive, illustrative embodiments thereof, these embodiments may be modified at will within the scope of the appended claims without departing from the spirit and nature of the present disclosure.

1. A tool manipulator, comprising:
a base, configured to be mounted on an operation table;
a caliper supported by the base;
a tool holder mounted on the caliper; and
an actuator positionable below a patient supporting surface of the operation table and configured to receive positioning commands for moving a tool in at least three degrees of freedom.

2. The tool manipulator of claim 1, wherein the actuator is a pneumatic actuator.

3. The tool manipulator of claim 2, wherein the at least three degrees of freedom comprise:
a first degree of freedom for moving the tool sideways in relation to the operation table;
a second degree of freedom for moving the tool vertically in relation to the operation table; and
a third degree of freedom for modifying a vertical angle of the tool.

4. The tool manipulator of claim 3, wherein the actuator is configured to moving the tool in four degrees of freedom, including a fourth degree of freedom for modifying a horizontal angle of the tool.

5. The tool manipulator of claim 4, wherein the actuator is configured to moving the tool in five degrees of freedom, including a fifth degree of freedom for moving the tool longitudinally in relation to the operation table.

6. The tool manipulator of claim 5, comprising:
a pair of cradles mounted on the base;
a pair of arms supporting the caliper, each arm being mounted on a respective cradle;
wherein the pneumatic actuator comprises:
first and second pneumatic cylinders independently operable to move the cradles sideways, whereby bringing the cradles closer to one another causes pivoting of the arms to raise the caliper and whereby moving both of the cradles in a same direction causes the caliper to move sideways; and
a third pneumatic cylinder operable to rotate the base horizontally;
a fourth pneumatic cylinder operable to rotate the base to modify a pitch of the caliper in relation to the patient supporting surface; and
a pair of fifth pneumatic cylinders operable to move the base horizontally along a length of the operation table.

7. The tool manipulator of claim 5, wherein the pneumatic actuator is operably connected to brackets supporting the caliper, the pneumatic actuator comprising:
a first pneumatic cylinder operable to move the base horizontally along a length of the operation table;
a second pneumatic cylinder operable to rotate the base horizontally; and
third, fourth and fifth pneumatic cylinders operable to move the brackets vertically in relation to the operation table;
wherein operation of the third and fourth pneumatic cylinders moves the caliper vertically in relation to the operation table, operation of the third and fifth pneumatic cylinders rotates the caliper for moving the tool to the left or to the right, and operation of the fourth and fifth pneumatic cylinder modifies a pitch of the caliper in relation to the patient supporting surface.

8. The tool manipulator of claim 2, comprising a pneumatic brake operably connected to the pneumatic actuator, the pneumatic brake being configured to prevent movements of the base and of the caliper when receiving a blocking command.

9. The tool manipulator of claim 5, comprising:
a pair of cradles mounted on the base;
a pair of arms supporting the caliper, each arm being mounted on a respective cradle;
wherein the pneumatic actuator comprises first and second step-by-step chariots independently operable to move the cradles sideways, whereby bringing the cradles closer to one another causes pivoting of the arms to raise the caliper and whereby moving both of the cradles in a same direction causes the caliper to move sideways.

10. The tool manipulator of claim 9, wherein the first and second step-by-step chariots ride on a pair of rails of the base and on an oscillation rod parallel to the pair of rails, the oscillating rod being configured to move back and forth along a direction of movement of the chariots, the chariots including pressure membranes adapted to solidarize the chariots, in successive steps, to the oscillating rod and to the pair of rails.

11. The tool manipulator of claim 1, comprising an optical detector of a position of the tool.

12. The tool manipulator of claim 11, wherein the optical detector detects the position of the tool over the at least three degrees of freedom.

13. The tool manipulator of claim 1, wherein the tool is a needle.

14. The tool manipulator of claim 13, wherein the tool manipulator is adapted to hold a needle configured for diagnosis or treatment of the prostate.

15. The tool manipulator of claim 14, further comprising a perineum conditioner configured to maintain in position a needle inserted in the perineum of a patient.

16. A system for positioning a needle for diagnosis or treatment of the prostate of a patient, comprising:
an operation table;
the tool manipulator of claim 1, the tool manipulator being adapted to support a needle and being integrated in the operation table;
a power source operably connected to the actuator; and
a controller operably connected to the power source and controlling provision of the positioning commands to the actuator.

17. The system of claim 16, comprising a hip positioner integrated within the patient supporting surface of the operation table and configured to adjust a height, an angle or both the height and angle of the hips of a patient lying on the patient supporting surface in relation to the tool manipulator.

18. The system of claim 16, wherein the actuator is a pneumatic actuator and wherein the power source is a pneumatic source.

19. The system of claim 18, comprising a pneumatic hip positioner integrated within the patient supporting surface of the operation table, operably connected to the pneumatic source, and configured to adjust a height, an angle or both the height and angle of the hips of a patient lying on the patient supporting surface in relation to the tool manipulator, the controller being further configured to control operation of the pneumatic hip positioner.

20. The system of claim 18, comprising a pneumatic connection between the pneumatic source and the pneumatic actuator, the pneumatic connection being routed via the operation table.

21. The system of claim 20, wherein the pneumatic connection is configured to transmit the positioning commands from the pneumatic source to the pneumatic actuator.

22. The system of claim 16, comprising a pair of footrests.

23. The system of claim 22, wherein the footrests are positionable in four degrees of freedom.

24. The system of claim 23, wherein the four degrees of freedom of the footrests include:
up and down adjustment of the footrests;
lengthwise adjustment of the footrests;
adjustment of a width between the footrests; and
rotational adjustment of the footrests.

25. The system of claim 16, comprising an integral leg support resting on the operation table.

26. The system of claim 16, comprising an optical fiber connection between the controller and the tool manipulator, the optical fiber connection being routed via the operation table.

27. The system of claim 26, wherein the optical fiber connection is configured to transmit positioning information from the tool manipulator to the controller.

28. The system of claim 16, comprising an operator console operably connected to the controller.

29. The system of claim 28, wherein the operator console is configured to control:
a positioning of the patient on the operation table;
a positioning of the tool manipulator;
an acquisition of image information from the prostate of the patient; and
a verification of the placement of one or more needles in relation to the prostate of the patient.

30. The system of claim 16, further comprising a medical imaging system.

31. The system of claim 30, wherein the medical imaging system is selected from the group consisting of a magnetic resonance imaging (MRI) system, a computerizing tomography (CT-scan) imaging system, an utrasonographic system, a positron emission tomographic system, a thermal imaging system, and a radiology system.

32. A step-by-step pneumatic system, comprising:
a frame supporting end membranes at each end of the frame, the frame further supporting a rail extending between the ends of the frame;
an oscillating rod mounted to the frame between the end membranes and parallel to the rail, the oscillating rod being configured to move back and forth along its longitudinal axis when pneumatic pressure is applied in one or the other of the end membranes in successive steps; and
a chariot riding on the rail and on the oscillating rod, the chariot including a coupling membrane adapted to solidarize the chariot to the oscillating rod and a braking membrane adapted to solidarize the chariot to the rail.

33. The step-by-step pneumatic system of claim 32, comprising a pneumatic control system adapted to:
apply pressure on the coupling membrane to solidarize the chariot to the oscillating rod;
apply pressure on a first end membrane, causing the oscillating rod and the chariot to move away from the first membrane;
apply pressure on the braking membrane;
release pressure on the coupling membrane;
release pressure on the first end membrane;
apply pressure on a second end membrane opposite from the first membrane, causing the oscillating rod to move toward the first membrane while the chariot remains in fixed position;
wherein the pneumatic control system is further adapted to repeat application of pressure of the membranes of the system until the chariot reaches a desired position.
